(19) United States
(12) Patent Application Publication
(10) Pub. No.: US 2026/0182898 A1
(43) Pub. Date: Jul. 2, 2026

(54) An Analysis and Identification Method, System and Storage Medium for Electrocardiogram

(71) Applicant: GUANGDONG PROVINCIAL PEOPLE'S HOSPITAL, Guanzhou (CN)

(72) Inventors: Huiying Liang, Guanzhou (CN); Yunmei Du, Guanzhou (CN); Lianting Hu, Guanzhou (CN); Shuai Huang, Guanzhou (CN); Huazhang Liu, Guanzhou (CN)

(21) Appl. No.: 18/024,677

(22) PCT Filed: Jan. 13, 2023
(86) PCT No.: PCT/CN2023/072195

§ 371 Date: Mar. 3, 2023

(30) Foreign Application Priority Data
Nov. 7, 2022 (CN) 202211390694.5

(51) Int. Cl. A61B 5/364; A61B 5/308; A61B 5/352; A61B 5/353; A61B 5/355; A61B 5/36; G16H 50/20; G16H 50/70

(52) U.S. Cl. CPC A61B 5/364; A61B 5/308; A61B 5/352; A61B 5/353; A61B 5/355; A61B 5/36; G16H 50/20; G16H 50/70

(57) ABSTRACT

The present invention discloses an analysis and identification method, system and storage medium for electrocardiograms. The method includes: constructing a concept tree based on definitions in electrocardiogram and arrhythmia knowledge, and establishing an arrhythmia electrocardiogram data set; performing data enhancement processing on the arrhythmia electrocardiogram data set based on the concept tree to construct prior data; constructing an ECG prior model based on the prior data; optimizing the ECG prior model to obtain a target identification model; and analyzing and identifying an electrocardiogram to be analyzed based on the target identification model, and determining an identification result of the electrocardiogram to be analyzed. The present invention has high accuracy and high efficiency, and can be widely used in the field of computer technologies.

### TECHNICAL FIELD

The present invention relates to the field of computer technologies, in particular to an analysis and recognition method and system for electrocardiogram and a storage medium.

### BACKGROUND ART

Arrhythmia is often accompanied by a series of clinical symptoms and complications, even life-threatening. With the aging of the population and the changes in lifestyle, the incidence of arrhythmia is rising rapidly, showing the trends of younger patients and growing incidence. An electrocardiogram (ECG) is a fundamental tool in medical practice; over 300 million ECGs are generated worldwide each year. It plays a key role in the process of diagnosing arrhythmias. ECG can relatively accurately reflect the nature and degree of arrhythmia. Thus, detecting cardiac arrhythmias with ECG recordings becomes a challenging yet significant task.

At present, the error rate of ECG machine interpretation is still very high. Clinically, ECG detection still relies on visual observation by experienced physicians. However, the manual interpretation method is limited by the skills and experiences of the professionals. In addition, the process is boring, time-consuming, inefficient, and lacks a unified objective standard, which may easily lead to misjudgments and omissions.

In recent years, the rapid development of deep learning enables computers to show near or even human-level cognitive abilities in many types of tasks. Deep learning is an algorithm in machine learning that models the multilayered representation of the underlying distribution of data. In other words, a deep learning algorithm can automatically extract low-level or high-level features needed for classification. Accordingly, deep learning can better represent the characteristics of data. At the same time, since the model has many levels and parameters, and the capacity is sufficient, a deep learning model has the ability to represent large-scale data. Therefore, for difficult problems such as images and voices that are not obvious, deep learning can be used to achieve better results on large-scale training data. Moreover, as deep learning can combine features and classifiers into one framework, and use data to learn features, it helps reduce the huge workload of manually extracting features in applications. Therefore, not only the effect therefrom can be better, but also it is very convenient to use.

### SUMMARY OF THE INVENTION

In light of the foregoing, the embodiments of the present invention provide a method and system for analyzing and identifying an electrocardiogram with high accuracy and high efficiency, and a storage medium.

An aspect of the embodiments of the present invention provides a method for analyzing and identifying an electrocardiogram, comprising:

An aspect of the embodiments of the present invention provides a method for analyzing and identifying an electrocardiogram, and the method includes the following steps:
constructing a concept tree based on definitions in electrocardiogram and arrhythmia knowledge, and establishing an arrhythmia electrocardiogram data set;
performing data enhancement processing on the arrhythmia electrocardiogram data set based on the concept tree to construct prior data;
constructing an ECG prior model based on the prior data;
optimizing the ECG prior model to obtain a target identification model; and
analyzing and identifying an electrocardiogram to be analyzed based on the target identification model, and determining an identification result of the electrocardiogram to be analyzed.

Optionally, the step of performing data enhancement processing on the arrhythmia electrocardiogram data set based on the concept tree to construct the prior data includes:
locating feature points in the ECG data;
segmenting P-QRS-T feature waves and segments based on the feature points obtained in the locating step;
determining control points of each waveform to form ECG features;
employing a K-means method to cluster the features of each ECG segment to form prior data; and
splicing the prior data of each fragment to obtain expanded and enhanced prior data.

Optionally, the step of constructing the ECG prior model based on the prior data includes a sub-step of constructing an ECG arrhythmia data set, and the sub-step includes:
extracting an ECG data samples;
screening and filtering the ECG data sample to obtain a training data set and a testing data set; and
performing data distribution and class similarity analysis on the training data set and the testing data set, so as to improve a discrimination degree of cardiac rhythm data in the training data set and the testing data set.

Optionally, the step of constructing the ECG prior model based on the prior data further includes a sub-step of preprocessing the prior data, and the sub-step includes:
removing myoelectric signals from the prior data by a Butterworth low-pass filter;
removing power frequency interference signals from the prior data by a 50 Hz finite impulse response notch filter with a Kaiser window function; and
removing ECG baseline shifts from the prior data by an infinite impulse response zero-phase shift digital filter.

Optionally, the step of segmenting the P-QRS-T feature waves and segments based on the feature points obtained in the locating step includes:
determining a position of an R peak by an R peak detection algorithm based on the feature points obtained in the locating step;
using a moving window of 250 ms to iteratively query both sides of the R peak, and determining a minimum value in a first window on the left as a position of a Q peak, and determining a minimum value in a first window on the right as an S peak;
performing time window traversal starting from a QRS complex to detect feature points of a P wave and a T wave based on the feature points obtained in the locating step;
decomposing an ECG signal into multiple periodic heartbeats based on detected key points;
dividing each heartbeat period is into 6 segments: a P wave, a P-Q interval, a QRS complex, an S-T interval, a T wave, and a T-P interval based on the feature points obtained in the locating step, wherein the P wave is defined as a signal between a P start point and a P end point;
specifically, the step of performing the time window traversal starting from the QRS complex to detect the feature points of the P wave and the T wave based on the feature points obtained in the locating step includes:
finding start points and end points of the P wave and the T wave by means of calculating a maximum distance between a start point and an end point of an auxiliary line segment at each point on the signal based on a boundary detection method of local distance transform;
establishing a 200 ms time window before a Q start point of the QRS complex, and establishing a 400 ms time window after an S end point; and
determining a P peak and a T peak using an algorithm similar to an R peak detection algorithm used for R peak detection, and using local distance transformation to determine the start points and end points of the P wave and the T wave.

Optionally, the step of determining the control points of each waveform to form ECG features includes:
separately determining a distribution of a length of each segment;
calculating an average length of each type of segment;
determining the number of control points and the number of indexes in each segment based on the average length thereof; and
calculating the control points in each segment and determining a corresponding ECG feature based on the average length, the number of control points and the number of indexes.

Optionally, the step of employing the K-means method to cluster the features of each ECG segment to form the prior data includes:
calculating a distance between an instance and a cluster centroid using an Euclidean distance;
configuring a number of clusters;
employing the K-means method to perform clustering for each type of segment represented by the control points thereof based on the distance and the number of clusters; and
upon finishing the clustering, assigning each segment with a cluster code based on a cluster class to which the segment belongs.

Optionally, the method further includes:
classifying a spliced ECG signal by means of classified naive Bayes based on an encoded feature.

Another aspect of the embodiments of the present invention further provides a system for analyzing and identifying an electrocardiogram, the system includes:
a first module, which is configured to construct a concept tree based on definitions in electrocardiogram and arrhythmia knowledge, and establish an arrhythmia electrocardiogram data set;
a second module, which is configured to perform data enhancement processing on the arrhythmia electrocardiogram data set based on the concept tree to construct prior data;
a third module, which is configured to construct an ECG prior model based on the prior data;
a fourth module, which is configured to optimize the ECG prior model to obtain a target identification model; and
a fifth module, which is configured to analyze and identify an electrocardiogram to be analyzed based on the target identification model, and determine an identification result of the electrocardiogram to be analyzed.

Another aspect of the embodiments of the present invention further provides a computer-readable storage medium, the storage medium stores a program, and the program can be executed by a processor to implement the method as described above.

The embodiments of the present invention further disclose a computer program product or a computer program. The computer program product or computer program includes a computer instruction(s). The computer instruction(s) is stored on the computer-readable storage medium. The processor of a computer device can read the computer instruction(s) from the computer-readable storage medium, and the processor can execute the computer instruction(s), so that the computer device executes the method as described above.

According to the embodiments of the present invention, a concept tree is constructed based on definitions of electrocardiogram and arrhythmia knowledge, and an arrhythmia electrocardiogram data set is established; data enhancement processing is performed with the arrhythmia electrocardiogram data set based on the concept tree to construct prior data; an ECG prior model is constructed based on the prior data; the ECG prior model is optimized to obtain a target identification model; based on the target recognition model, an electrocardiogram to be analyzed is analyzed and identified, and an identification result of the electrocardiogram to be analyzed is then determined. The present invention has high accuracy and high efficiency.

### BRIEF DESCRIPTION OF THE DRAWINGS

In order to more clearly illustrate the technical solutions in the embodiments of the present application, the following will briefly introduce the drawings for the description of the embodiments. Obviously, the accompanying drawings in the following description are only some embodiments of the present application. For a person skilled in the art, other drawings may also be obtained based on the drawings provided herein without creative efforts.

FIG. 1 is an overall technical roadmap provided by the embodiments of the present invention.

FIG. 2 is a processing flowchart of an arrhythmia data set provided by the embodiments of the present invention.

FIG. 3 is a schematic diagram of different heartbeat positioning points provided by the embodiments of the present invention.

FIG. 4 is a flow chart of heartbeat splicing and encoding provided by the embodiments of the present invention.

FIG. 5 is a flow chart of the overall steps of the embodiments of the present invention.

### DESCRIPTION OF THE EMBODIMENTS

In order to make the purposes, technical solutions and advantages of the present application clearer, the present application will be further described in detail below in conjunction with the accompanying drawings and embodiments. It should be understood that the specific embodiments described herein are only used to explain the present application, and are not intended to limit the present application.

### Terminology

There are following 18 types of ECG categories that can be identified by the classification provided in the present invention:

### Tachycardia and Bradycardia:

1. Atrial flutter (AFL)
2. Atrial fibrillation (AF)
3. Ventricular tachycardia (VT)
4. Atrial tachycardia (AT)
5. Sinus tachycardia (ST)
6. Atrioventricular junction tachycardia (JT)
7. Sinus bradycardia (SB)
8. Sinus arrhythmia (SA)

### Conduction Abnormalities:

9. Sinoatrial conduction block (SA block)
10. Intraventricular conduction block (IV block)
11. Atrioventricular conduction block (AV block)

### Origin Abnormalities:

12. Premature atrial contraction (PAC)
13. Premature ventricular contraction (PVC)
14. Atrioventricular junctional premature contraction (PJC)
15. Ventricular escape (VE)
16. Atrioventricular junction escape (JE)
17. Atrioventricular junction escape rhythm (JER)
18. Normal

Electrocardiogram (ECG) is a technology that uses an electrocardiograph to record the changes in electrical activity of the heart during each cardiac cycle from the body surface. It reflects a series of changes in the occurrence, propagation and recovery process of cardiac excitation.

The waves and segments in the composition of an electrocardiogram are as follows: P wave: The electrical excitation of the normal heart begins at the sinoatrial node. Since the sinoatrial node is located at the junction between the right atrium and the superior vena cava, the excitation of the sinoatrial node is first transmitted to the right atrium, and then transmitted to the left atrium through the interatrial bundle, so as to form the P wave in the ECG. The P wave represents the activation of the atrium; the first half thereof represents the activation of the right atrium, and the second half thereof represents the activation of the left atrium. The duration of the P wave is 0.12 seconds and the height thereof is 0.25 mv. When the atrium is enlarged and the conduction between the two atria is abnormal, the P wave can appear as a high-pointed or double-peaked P wave.

PR interval: The PR interval represents the time required for the excitation generated by the sinoatrial node to reach the ventricle by way of the atrium, the atrioventricular junction and the atrioventricular bundle, which then causes the ventricular muscle to start exciting. Therefore, it is also referred to as the atrioventricular conduction time. The normal PR interval is typically between 0.12 seconds and 0.20 seconds. When the conduction from the atrium to the ventricle is blocked, it can be manifested as a prolongation of the PR interval or the disappearance of the ventricular wave after the P wave.

QRS complex: The excitation is transmitted downward through the His bundle, and the left and right bundle branches to further excite the left and right ventricles synchronously, forming the QRS complex. The QRS complex represents the depolarization of the ventricles with the excitation duration of less than 0.11 seconds. When there is conduction block of the left and right bundle branches of the heart and in the cases of ventricular enlargement or hypertrophy, etc., the QRS complex can be widened, deformed and prolonged.

Point J: The intersection point where the QRS wave ends and the ST segment begins. It represents the complete depolarization of ventricular myocytes.

ST segment: a period of time when all depolarization of the ventricular muscles is complete and repolarization has not yet begun. At this time, the ventricular muscles in all parts are in a state of depolarization, and there is no potential difference between cells. Therefore, under normal circumstances, the ST segment should be on the equipotential line. When there is ischemia or necrosis in a certain part of the myocardium, there can still be a potential difference in the ventricle after the depolarization is completed. In such a case, it is manifested as a shift of the ST segment on the electrocardiogram.

T wave: The subsequent T wave represents the repolarization of the ventricles. In leads where the dominant QRS wave is upward, the T wave should be in the same direction as the dominant QRS wave. The change of T wave on ECG can be the result from many factors. For example, myocardial ischemia can be manifested as low flat inversion of T wave. The towering T wave can be seen in hyperkalemia, the hyperacute phase of acute myocardial infarction, and the like.

U wave: U wave can be seen after the T wave in some leads, which is currently considered to be related to the repolarization of the ventricle.

QT interval: It represents the time period from depolarization to repolarization of the ventricles. The normal QT interval is 0.44 seconds. Since the QT interval can be influenced by heart rate, the concept of the corrected QT interval (QTC) was further introduced. One calculation method is QTc=QT/√RR. Prolongation of the QT interval is often associated with the occurrence of malignant arrhythmias.

ECG leads: The heart is a three-dimensional structure. Thus, in order to reflect the electrical activity of different sides of the heart, electrodes can be placed in different parts of the human body to record and reflect the electrical activity of the heart. During a routine ECG examination, usually only 4 limb lead electrodes and V₁ to V₆ of 6 chest lead electrodes are placed; thus the conventional 12-lead ECG is recorded.

Cardiac conduction system: The cardiac conduction system is composed of special myocardial cells that can generate and conduct impulses in the myocardium, including the sinoatrial node, the endo-intermediate bundle, the atrioventricular node, the atrioventricular bundle, the right bundle branch, the left bundle branch, and the Purkinje fibers. The sinoatrial node is the pacemaker of the normal heart rate, located below the epicardium between the entrance of the superior vena cava and the right atrial appendage. The conjunctival bundle is the conduction pathway between the sinoatrial node and the atrioventricular node. It can be divided into three conduction bundles: the anterior conjunctival fascicle, the middle conjunctival fascicle and the posterior conjunctival fascicle, where the anterior interstitial bundle further forms a branch to the left atrium, which is referred to as the interatrial bundle. The atrioventricular node is located below the endocardium on the right side of the atrial septum, lying across the area between the ostium of the coronary sinus, the fossa ovale, and the upper border of the tricuspid septal valve, extending downward into the atrioventricular bundle. The atrioventricular node and the atrioventricular bundle (His bundle) constitute the atrioventricular junction area, and then extend forward and downward to the lower end of the interventricular septum, where each is further divided into left and right bundle branches, which are respectively located below the left and right endocardium of the interventricular septum. The left bundle branch is at the beginning of the left side of the interventricular septum, and is divided into two bundles of fibers, the anterior branch and the superior branch. The right bundle branch descends along the right side of the interventricular septum until it begins to branch into Purkinje fibers at the apex of the heart.

The right bundle branch connects with the Purkinje fibers below the endocardium and finally connects with the ventricular muscle.

The function of the cardiac conduction system is to generate impulses and conduct them to all parts of the heart, so that the atrial and ventricular muscles contract according to a certain rhythm.

1) The conduction system of the heart includes the sinoatrial node, the atrioventricular node, the atrioventricular bundle, the branches of the left and right atrioventricular bundles, and many fine branches distributed to the ventricular papillary muscle and the ventricular wall.

2) Except that the sinoatrial node is located in the deep epicardium of the right atrium, the rest are distributed in the subendocardium.

3) There are three types of special myocardial fibers that make up the conduction system of the heart: pacemaker cells (participating in the formation of the sinoatrial node and atrioventricular node), transitional cells (acting as conduction impulses), and Purkinje fibers (capable of rapidly transmitting impulses).

4) The Purkinje fibers at the end of the branch of the atrioventricular bundle are connected with the ventricular muscle.

5) The function of the cardiac conduction system is to generate and conduct impulses to maintain the rhythmic beating of the heart.

Arrhythmia is due to abnormal sinoatrial node excitation or excitation outside the sinoatrial node, and the conduction of the excitation is slow, blocked, or conducted through abnormal channels, that is, the origin of cardiac activity and/or conduction disorders leading to abnormalities in the frequency of cardiac beating and/or rhythm. Arrhythmia is an important group of cardiovascular diseases. It may occur alone or with other cardiovascular diseases. Its prognosis is related to the etiology, inducement, evolution trend of arrhythmia, and whether it leads to severe hemodynamic disturbance. It can attack suddenly and cause sudden death, or it may continue to affect the heart and cause it to fail finally.

7. Arrhythmia includes the following five categories:

\textcircled{1} Premature beat: Premature beat is also referred to as premature contraction. The premature beat is the heartbeat caused by premature impulses in one part of the heart. According to the location of occurrence, it can be divided into atrial premature contraction, junctional premature contraction, and ventricular premature contraction. Premature contraction may not cause symptoms, if there is no structural heart disease, and the prognosis thereof can be good. Some patients may have palpitations, dizziness, fatigue, and the like. It can be treated symptomatically, and if there is an organic heart disease, the underlying heart disease should be treated.

\textcircled{2} Atrial flutter and Atrial fibrillation: In atrial flutter, the atrial rate is usually from 220 to 360 beats/min, and generally cannot be fully transmitted to the ventricle. Due to physiological atrioventricular block, 2:1 or 3:1 downlink can be formed, and occasionally 1:1 atrioventricular conduction may also occur. Atrial fibrillation is a rapid cardiac arrhythmia with multifocal microreentry in the atrium. The frequency can be from 350 to 600 beats/min, and the ventricular arrhythmia can be 120 to 160 beats/min. Atrial flutter and atrial fibrillation are common in rheumatic heart disease, hyperthyroidism, coronary heart disease, cardiomyopathy and hypertensive heart disease. The etiology of many patients with atrial fibrillation is unknown.

\textcircled{3} Supraventricular paroxysmal tachycardia: It is a paroxysmal rapid and regular ectopic rhythm. The heart rate is generally from 160 to 220 beats/min, but there are also those cases which are as slow as 130 beats/min or as fast as 300 beats/min. According to the mechanism of occurrence, it can be divided into three types: atrial, atrioventricular nodal reentry and atrioventricular bypass reentry. It is common in the cases without structural heart disease, and the etiology is unknown. It can also be seen in rheumatic heart disease, cardiomyopathy, coronary heart disease, and the like. The clinical manifestations include sudden onset, which may last for several seconds, minutes to hours, or even after several days and then stops suddenly. Severe attacks can cause insufficient blood supply to the heart and brain, leading to a drop in blood pressure, dizziness, nausea, angina pectoris, or fainting.

\textcircled{4} Ventricular tachycardia and ventricular fibrillation: Cases of than 3 consecutive premature ventricular beats are referred to as ventricular tachycardia, which is more common in patients with structural heart disease. Sustained ventricular tachycardia refers to those cases that last for more than 30 seconds or severe hemodynamic disturbance occurs within 30 seconds, and non-sustained ventricular tachycardia refers to those cases that terminate spontaneously within 30 seconds. Torsade de ventricular tachycardia is a special type of ventricular tachycardia, which is more common in long QT syndrome. It can be divided into two types: congenital and acquired. Ventricular tachycardia can turn into ventricular fibrillation if left untreated. Ventricular fibrillation is the most serious arrhythmia and requires immediate electrical defibrillation to convert the heart rhythm.

\textcircled{5} Bradycardia: Adults with a heart rate below 60 beats per minute are referred to as bradycardia, which can be caused by sick sinus syndrome or atrioventricular block.

The mechanism of arrhythmia includes the following two types:

### 1) Abnormalities in Impulse Formation:

Cardiomyocytes in the sinoatrial node, the bundles between the junctions, the location near the ostium of the coronary sinus, the distal end of the atrioventricular node, and the bundle of the Hirsch-Purkinje system are autonomic (pacemaker) cells. Altered autonomic nervous system excitability or intrinsic pathology can lead to inappropriate impulse firing. In addition, cardiomyocytes that originally had no pacemaker property, such as atrial and ventricular cells, can also appear abnormally as pacemaker cells in certain pathological conditions, for example, myocardial ischemia, drugs, electrolyte disturbances, and increased catecholamines can all lead to the formation of abnormal automaticity.

### 2). Abnormalities in Impulse Conduction:

Reentry is the most common mechanism for all tachyarrhythmias.

According to the mechanism of occurrence, the classification of arrhythmia includes the following two categories:

### 1. Abnormalities in Impulse Formation:

1) Sinus arrhythmia

(1) sinus tachycardia, (2) sinus bradycardia, (3) sinus arrhythmia, and (4) sinus arrest

2) Ectopic cardiac rhythm

\textcircled{1} Passive ectopic cardiac rhythm (1) Escape (atrial, atrioventricular junctional, ventricular); (2) paroxysmal tachycardia (atrial, atrioventricular junctional, ventricular)

\textcircled{2} Active ectopic cardiac rhythm (1) premature systole (atrial, atrioventricular junctional, ventricular); (2) paroxysmal tachycardia (atrial, atrioventricular junctional, atrioventricular reentrant, ventricular); (3) atrial flutter, atrial fibrillation; (4) ventricular flutter, ventricular fibrillation

### 2. Abnormalities in Impulse Conduction:

1) Physiological interference and atrioventricular separation

2) Pathological conditions: (1) sinoatrial block; (2) intraatrial block; (3) atrioventricular block; (4) bundle branch or branch block (block in the left bundle branch, the right bundle branch, or the branches of the left bundle branch), or intraventricular block

3) Abnormal pre-excitation syndrome of atrioventricular conduction pathway

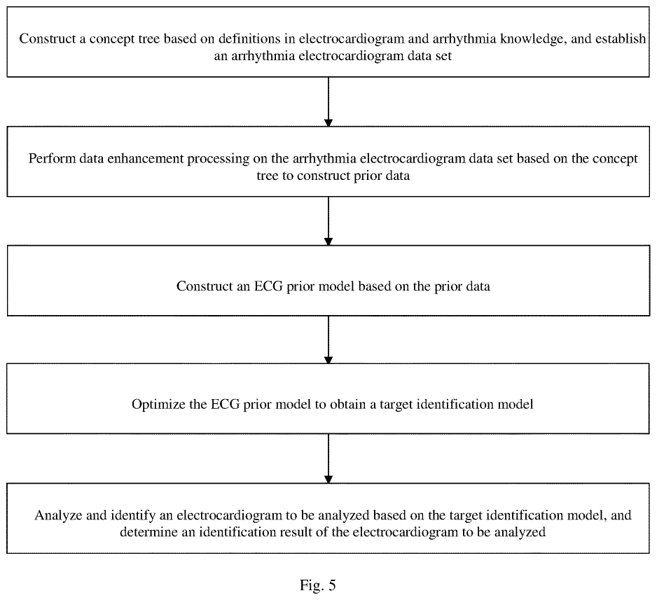

At present, for the analysis method of electrocardiogram, the manual interpretation method is limited by the skills and experience of the professionals; the process is boring, time-consuming and inefficient. In view of the problems mentioned above in the existing technologies, an aspect of the embodiments of the present invention provides a method for analyzing and identifying an electrocardiogram. As shown in FIG. 5, the method includes the following steps:
constructing a concept tree based on definitions in electrocardiogram and arrhythmia knowledge, and establishing an arrhythmia electrocardiogram data set;
performing data enhancement processing on the arrhythmia electrocardiogram data set based on the concept tree to construct prior data;
constructing an ECG prior model based on the prior data;
optimizing the ECG prior model to obtain a target identification model; and
analyzing and identifying an electrocardiogram to be analyzed based on the target identification model, and determining an identification result of the electrocardiogram to be analyzed.

Optionally, the step of performing data enhancement processing on the arrhythmia electrocardiogram data set based on the concept tree to construct the prior data includes:
locating feature points in the ECG data;
segmenting P-QRS-T feature waves and segments based on the feature points obtained in the locating step;
determining control points of each waveform to form ECG features;
employing a K-means method to cluster the features of each ECG segment to form prior data; and
splicing the prior data of each fragment to obtain expanded and enhanced prior data.

Optionally, the step of constructing the ECG prior model based on the prior data includes a sub-step of constructing an ECG arrhythmia data set, and the sub-step includes:
extracting an ECG data samples;
screening and filtering the ECG data sample to obtain a training data set and a testing data set; and
performing data distribution and class similarity analysis on the training data set and the testing data set, so as to improve a discrimination degree of cardiac rhythm data in the training data set and the testing data set.

Optionally, the step of constructing the ECG prior model based on the prior data further includes a sub-step of preprocessing the prior data, and the sub-step includes:
removing myoelectric signals from the prior data by a Butterworth low-pass filter;
removing power frequency interference signals from the prior data by a 50 Hz finite impulse response notch filter with a Kaiser window function; and
removing ECG baseline shifts from the prior data by an infinite impulse response zero-phase shift digital filter.

Optionally, the step of segmenting the P-QRS-T feature waves and segments based on the feature points obtained in the locating step includes:
determining a position of an R peak by an R peak detection algorithm based on the feature points obtained in the locating step;
using a moving window of 250 ms to iteratively query both sides of the R peak, and determining a minimum value in a first window on the left as a position of a Q peak, and determining a minimum value in a first window on the right as an S peak;
performing time window traversal starting from a QRS complex to detect feature points of a P wave and a T wave based on the feature points obtained in the locating step;
decomposing an ECG signal into multiple periodic heartbeats based on detected key points;
dividing each heartbeat period is into 6 segments: a P wave, a P-Q interval, a QRS complex, an S-T interval, a T wave, and a T-P interval based on the feature points obtained in the locating step, wherein the P wave is defined as a signal between a P start point and a P end point;
specifically, the step of performing the time window traversal starting from the QRS complex to detect the feature points of the P wave and the T wave based on the feature points obtained in the locating step includes:
finding start points and end points of the P wave and the T wave by means of calculating a maximum distance between a start point and an end point of an auxiliary line segment at each point on the signal based on a boundary detection method of local distance transform;
establishing a 200 ms time window before a Q start point of the QRS complex, and establishing a 400 ms time window after an S end point; and
determining a P peak and a T peak using an algorithm similar to an R peak detection algorithm used for R peak detection, and using local distance transformation to determine the start points and end points of the P wave and the T wave.

Optionally, the step of determining the control points of each waveform to form ECG features includes:
separately determining a distribution of a length of each segment;
calculating an average length of each type of segment;
determining the number of control points and the number of indexes in each segment based on the average length thereof; and
calculating the control points in each segment and determining a corresponding ECG feature based on the average length, the number of control points and the number of indexes.

Optionally, the step of employing the K-means method to cluster the features of each ECG segment to form the prior data includes:
calculating a distance between an instance and a cluster centroid using an Euclidean distance;
configuring a number of clusters;
employing the K-means method to perform clustering for each type of segment represented by the control points thereof based on the distance and the number of clusters; and
upon finishing the clustering, assigning each segment with a cluster code based on a cluster class to which the segment belongs.

Optionally, the method further includes:
classifying a spliced ECG signal by means of classified naive Bayes based on an encoded feature.

Another aspect of the embodiments of the present invention further provides a system for analyzing and identifying an electrocardiogram, the system includes:
a first module, which is configured to construct a concept tree based on definitions in electrocardiogram and arrhythmia knowledge, and establish an arrhythmia electrocardiogram data set;
a second module, which is configured to perform data enhancement processing on the arrhythmia electrocardiogram data set based on the concept tree to construct prior data;
a third module, which is configured to construct an ECG prior model based on the prior data;
a fourth module, which is configured to optimize the ECG prior model to obtain a target identification model; and
a fifth module, which is configured to analyze and identify an electrocardiogram to be analyzed based on the target identification model, and determine an identification result of the electrocardiogram to be analyzed.

Another aspect of the embodiments of the present invention further provides a computer-readable storage medium, the storage medium stores a program, and the program can be executed by a processor to implement the method as described above.

The embodiments of the present invention further disclose a computer program product or a computer program. The computer program product or computer program includes a computer instruction(s). The computer instruction(s) is stored on the computer-readable storage medium. The processor of a computer device can read the computer instruction(s) from the computer-readable storage medium, and the processor can execute the computer instruction(s), so that the computer device executes the method as described above.

The specific implementation process of the present invention will be described in detail below in conjunction with the accompanying drawings:

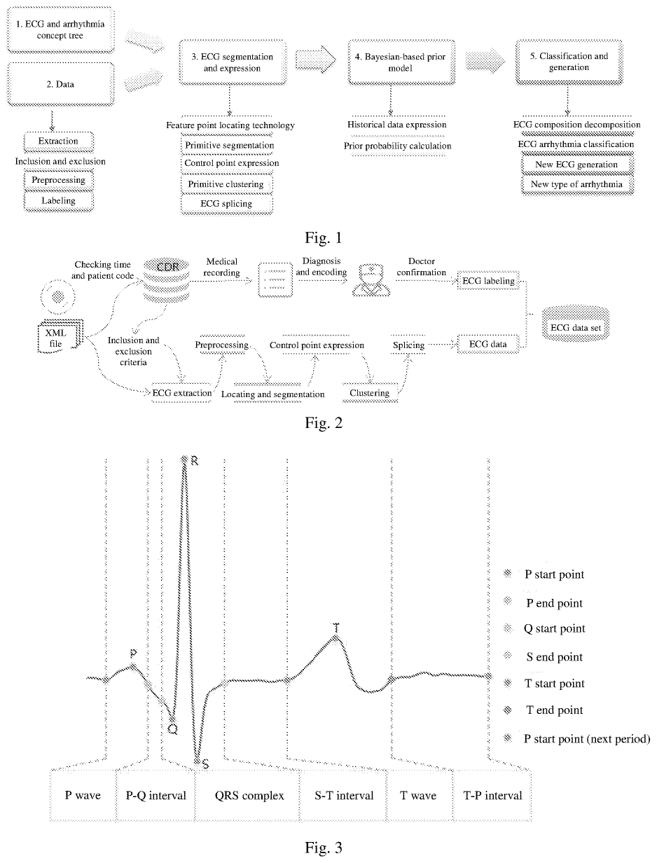

As shown in FIG. 1, the overall implementation process of the present invention includes 5 major steps:

1. Concept tree of electrocardiogram and arrhythmia: a concept tree is formed by means of sorting out and redefining the knowledge of electrocardiogram and arrhythmia.

2. Data: an arrhythmia ECG data set is constructed through an innovative preprocessing method.

3. Segmentation and expression:
a) Feature points are located in the electrogram data by way of a novel home-made method, and P-QRS-T feature waves and segments are identified by means of segmentation based on the feature points;
b) The control point(s) of each waveform is determined to form ECG features;
c) A K-means method is used to cluster the data segments to form prior data;
d) The data fragments are spliced to expand and enhance the prior data.

4. Prior model: An ECG prior model based on the Bayesian theory is established.

5. Application and deduction of the priori model: On the basis of the ECG prior model, deduction is performed and inferences are drawn about other cases from one instance, so as to achieve small sample learning, and the decomposition and generation of ECG.

The specific implementation process of each step will be described in detail below:

### 1. ECG and Arrhythmia Concept Tree:

In the embodiments of the present invention, the categories of arrhythmia are systematically sorted out to form an arrhythmia concept tree. It can be understood that the arrhythmia concept tree can be categorized based on the specific location where the arrhythmia occurs. The present invention is capable of providing diagnostic classification of 18 ECG categories.

### 2. GDPH ECG Arrhythmia Data Set

The process of data labeling, processing and expression according to the embodiments of the present invention is shown in FIG. 2. The upper part of FIG. 2 is the process of classification and labeling with the samples; the lower part thereof is the process of processing and expressing the ECG data. The ECG labeling and the ECG data together constitute the data set that will be used by the ECG post-model.

### 2.1. Data Sample Extraction

In the present invention, ECG data have been obtained from the databases of related hospital. In addition, the ECG data of each subject have been labeled as normal or one of 17 types of arrhythmias. The 17 types of arrhythmias are as follows: premature ventricular contraction (PVC), intraventricular block (IV block), ventricular tachycardia (VT), ventricular escape (VE), atrial flutter (AFL), atrial tachycardia (AT), atrial fibrillation (AF), atrial premature contraction (PAC), junctional premature contraction (PJC), junctional escape (JE), junctional tachycardia (JT), junctional escape rhythm (JER), atrioventricular conduction block (AV block), sinoatrial block (SA block), sinus tachycardia (ST), sinus bradycardia (SB), sinus arrhythmia (SA). Moreover, based on the location of occurrence, the 17 types of arrhythmias can be divided into 4 supercategories: sinus (including SA block, ST, SB and SA), atrium (including AFL, AT, AF and PAC), atrioventricular node (including PJC, JE, JT, JER and AV block), and ventricle (including PVC, IV block, VT and VE).

### 2.2. Inclusion and Exclusion Criteria

The embodiments of the present invention exclude some ECG cases due to the following reasons: (1) ECG cases are severely distorted due to missing signal or excessive noise. Since an ECG records the heart's electrical activity by means of the electrodes placed on the skin, large movements and noisy surroundings can add irreducible noise to the signal. Furthermore, this distortion is more common in children's ECGs. (2) The labels of ECG cases are not available or uncertain. (3) For participants with multiple ECG tests, in order to avoid introducing bias to the population of participants, our study only uses the last ECG test results and excludes other ECG tests. Finally, 48,063 participants are excluded. The entire data set is randomly divided into a training data set and a testing data set at the label level with a ratio of 4:1.

In addition, the embodiments of the present invention also perform data distribution and class similarity analysis on the constructed training data set and testing data set. As the data sets are obtained from the cases in the real world, the GDPH ECG arrhythmia data set is extremely unbalanced. ST had the largest sample size (N=20273), and VE has the smallest sample size (N=10). The sample size standard deviation for all arrhythmia types is 5249.63. The mean and median sample sizes are 2670 and 106, respectively. An imbalance in the data set in sample size may prevent the model from learning all arrhythmia categories equally.

In the embodiments of the present invention, by means of constructing a category-level similarity matrix, each element in the matrix is the similarity between two arrhythmia categories. The category-level similarity is the average similarity of all ECG signal pairs from two arrhythmia categories respectively. Dynamic time warping (DTW) is used to measure the similarity between two ECG signals. The similarity matrix is normalized to be in the range [0, 1] and sorting is performed from the smallest to the largest. Arrhythmias are not evenly distributed in the representation space. SB, JER, SA, JE, AV block, PJC, PVC, AT, and AF are relatively similar. Those similar types of arrhythmia might be better at fooling the model. However, other arrhythmia types are more diffused and easier to distinguish.

### 2.3. ECG Preprocessing

The ECG signal is a weak physiological signal, which can be easily disturbed during the acquisition process. Therefore, ECG signals need to be preprocessed prior to analysis. The three most common disturbances to ECG signals are electromyography (EMG) disturbances, power frequency disturbances, and baseline drift. The preprocessing in the embodiments of the present invention is performed based on the above three types of interferences.

EMG signal is also a kind of physiological signal, and is the main noise in ECG signals. The frequency of the EMG signal is related to the type of muscle, generally in the range of 30 to 300 Hz, while the frequency of the ECG signal is mainly in the range of 5 to 20 Hz. Therefore, EMG signals can overlap with ECG signals. In our study, a Butterworth low-pass filter is used to remove EMG. The Butterworth low-pass filter has the flattest band-pass frequency response curve that taper to zero as the stop band are adjusted. In addition, the amplitude at the diagonal frequencies decreases monotonically; the higher the filter order, the faster the amplitude decays in the stop band.

Due to the existence of power supply networks, power frequency interference is inevitable. Interference signals with a frequency of 50 Hz are the most common interference signals. In this study, a 50 Hz finite impulse response (FIR) notch filter with the Kaiser window function is used to eliminate the power frequency signal. The FIR filter has the linear phase characteristics required for ECG signal processing, and can obtain the best filtering performance with the least waveform distortion. In addition, considering the overall difference of samples, the Kaiser moving window is employed, which is a window function close to the optimal structure, and can adaptively adjust the parameters of the filter based on different parameters.

Finally, ECG baseline drift is eliminated by an infinite impulse response zero-phase shift digital filter. As a routine preprocessing step in ECG analysis, it can prevent the introduction of artifact information that may distort the true oscillation phase. After preprocessing, the main noise is removed, while the key information of the ECG signal is preserved.

### 2.4 P-QRS-T Positioning and Segmentation

In the embodiments of the present invention, feature points of P-QRS-T and the like in the ECG data are located, and segments are then identified based on the feature points. This process mainly includes the following steps:

### 2.4.1. Locating P-QRS-T in ECG Data

This embodiment uses an adaptive and efficient R-peak detection algorithm to determine the position of the R-peak. Next, the Q and S peaks are further determined by searching on both sides of the R peak. Since multiple peaks are rarely present in the QRS complex, a moving window of 250 ms is used to iterate on both sides. The minimum value in the first window on the left side is the location of the Q peak. The minimum value of the first window on the right side is the S peak.

The detection of the P and T waves requires time window traversal starting from the QRS complex. Therefore, a detection algorithm for the start and end boundaries of waves is needed herein. To this end, this embodiment proposes an improved boundary detection algorithm based on local distance transformation. The local distance transform can find the start and end of waves by calculating the maximum distance between the start point and end point of an auxiliary line segment at each point on the signal. From a morphological point of view, the aforementioned point is the location with the greatest curvature, which is in line with the subjective judgment of a doctor.

A 200 ms time window is established before the Q start point, and a 400 ms time window is established after the S end point. The P peak and T peak are detected using the same detection algorithm as that used in the R peak detection. A local distance transform is also used herein to determine the start point and end point of the P wave and the start point and end point of the T wave.

FIG. 3 shows an example. Each heartbeat is divided into 6 segments by 11 points. A total of 11 points are identified for each heartbeat, which are as follows: a P start point, a P point, a P end point, a Q start point, a Q point, an R point, an S point, an S end point, a T start point, a T point, and a T end point. The division of each heartbeat and the division of heartbeat internal waves are based on the location of these feature points.

### 2.4.2. Heartbeat Period Determination and Heartbeat Period Segment Determination

Based on the key points detected in the step mentioned above, this embodiment decomposes the ECG signal into multiple periodic heartbeats. The entire data set of this embodiment has 459818 heartbeats in total. The number of ECG samples and the number of heartbeat periods for each category are shown in Table 1 below. Table 1 shows the number of heartbeats in each category from decomposing the ECG signal.

TABLE 1

| Category | Number of samples | Number of heartbeats |
|---|---|---|
| PAC | 1287 | 12751 |
| AF | 33 | 335 |
| SA | 9956 | 95233 |
| Av block | 483 | 4527 |
| JER | 131 | 993 |
| JT | 19 | 196 |
| VE | 9 | 76 |
| JE | 33 | 284 |
| Typical | 7944 | 75675 |
| SB | 1623 | 10464 |
| AT | 80 | 793 |
| VT | 63 | 624 |
| IV block | 3744 | 39050 |
| ST | 20272 | 195493 |
| AF | 64 | 653 |
| PJC | 64 | 670 |
| SA block | 10 | 95 |
| PVC | 2230 | 21906 |
| Total | 48045 | 459818 |

Each heartbeat period is further divided to the following 6 segments according to the locating points: a P wave, a P-Q interval, a QRS complex, a S-T interval, a T wave, and a T-P interval. The P wave is defined as the signal between the P start point and the P end point. In the same way, as shown in FIG. 7, other segments are also defined accordingly. Thus, each heartbeat corresponds to 6 segments. As a result, the number of instances of each segment is also 459818, which is equal to the number of samples of the heartbeats.

### 2.5. Determine the Control Points of Each Waveform to Form ECG Features:

This embodiment extracts control points for each segment; and then the extracted control points and corresponding indices thereof are used as representations for each segment.

First, the distributions of the lengths of the six segments are obtained respectively;

Next, the average length of each type of segment is calculated.

Suppose Lₘ (m=1, 2, . . . , M) represents the length of the m^{th} instance, where M is the total number of instances. The average length \hat{l} of the segment can be obtained by the following equation:

$$\hat{l} = \frac{1}{M} {\sum }_{m = 1}^{M} {l}_{m} \qquad \# ( 1 )$$

A control point is taken for every five points of the average length. As a result, the number of control points N extracted by this type of segment is as follows:

$$N = \frac{1}{5} \hat{l} \qquad ( 2 )$$

For each instance, the control points are extracted at equal intervals. Therefore, the n^{th} control point of the m^{th} instance can be obtained by the following equation:

$${C}_{n}^{m} = s i {g}^{m} ( \frac{n}{N} {l}_{m} ) \qquad ( 3 )$$

In the above equation,

$${C}_{n}^{M}$$

represents the value of the n^{th} control point in the m^{th} instance; sig^{m}(*) represents the signal value of the m^{th} instance whose index is *;

$$* ; \frac{n}{N} {l}_{m}$$

represents the corresponding index value of the n^{th} control point.

The average length, the number of control points and the number of indexes of each segment are shown in Table 2 below. That is, 12 control points are extracted for each P wave, 6 control points are extracted for each P-Q interval, 20 control points are extracted for each QRS wave, 6 control points are extracted for each S-T interval, 14 control points are extracted for each T-wave, and 18 control points are extracted for each T-P interval. Thus, 82 control points are extracted for each heartbeat period.

TABLE 2

| | P | P-Q | QRS | S-T | T | T-P |
|---|---|---|---|---|---|---|
| Segment | wave | interval | wave | interval | wave | interval |
| Length | 57.60 ± | 30.44 ± | 97.94 ± | 29.84 ± | 71.54 ± | 90.69 ± |
| (mean ± | 25.89 | 42.12 | 35.82 | 36.78 | 33.49 | 62.85 |
| variance) | | | | | | |
| Number of | 12 | 6 | 20 | 6 | 14 | 18 |
| control | | | | | | |
| points | | | | | | |
| extracted | | | | | | |
| Number of | 12 | 6 | 20 | 6 | 14 | 18 |
| indices | | | | | | |
| extracted | | | | | | |
| Number of | 24 | 12 | 40 | 12 | 28 | 36 |
| features | | | | | | |

There are 459818 heartbeat periods in the data set. Each period has 6 segments. There are 459818*6 instances in total. The index and the ECG signal value thereof are calculated separately for each instance. Thus, finally 459818*82 pairs of control points (index, ECG signal value) are obtained.

### 2.6 Segment Clustering

For each type of segment represented by the control points, a K-means method is used to cluster the segments to form prior data.

The distance between an instance and the cluster centroid is then measured using the Euclidean distance. The algorithm stops when the centroid does not change significantly over iterations. A total of 54 (=6 segments×9 leads) clusters need to be implemented for the 9 leads of 6 types of segments. In this example, the number of clusters is one of the hyperparameters. It has 18 options: 3, 5, 8, 10, 15, 20, 25, 30, 35, 40, 45, 50, 55, 60, 65, 70, 75, and 80.

In the embodiments of the present invention, the number of clusters is set to 25. After the number of clusters is determined, when the clustering is completed, each segment is assigned a cluster code according to the cluster category to which the segment belongs.

### 2.7. Splicing and Enhancement

In this section, the data fragments are spliced to expand the prior data.

### 2.7.1. Heartbeat Splicing and Encoding

In this embodiment, multiple original heartbeats are spliced together as a spliced ECG signal. The spliced ECG signal can be viewed as a permutation of the original heartbeat, allowing repetitions in the permutation. The spliced ECG signal can simulate an ECG signal that does not exist in the training data set, further enhancing the comprehensiveness of the training data set.

Suppose

$${N}_{j}^{i} ( i = 1 , 2 , … , C ; j = 1 , 2 , … , {M}_{i} )$$

represents the j^{th} original heartbeat number in the i^{th} ECG signal, where C is the total number of ECG categories, Mᵢ represents the number of heartbeats in the i^{th} ECG category, and Nₒ represents the number of original heartbeats to be spliced. Thus, for the j^{th} ECG signal in the i^{th} category, the number of ECG signals that can be can be obtained by splicing can be determined by the following equation:

$${\hat{N}}_{j}^{i} = {( {N}_{j}^{i} )}^{{N}_{o}} \qquad ( 4 )$$

Hence, the total number of spliced ECG signals N₀ is as follows:

$$\hat{N} = {\sum }_{i = 1}^{C} {\sum }_{i = 1}^{{M}_{i}} {\hat{N}}_{j}^{i} \qquad ( 5 )$$

Formula (4) is then introduced to formula (5), and the following equation can be obtained:

$$\hat{N} = {\sum }_{i = 1}^{C} {\sum }_{j = 1}^{{M}_{i}} {( {N}_{j}^{i} )}^{{N}_{o}} \qquad ( 6 )$$

In this embodiment, Nₒ is another hyperparameter controlling \hat{N}. It has 4 options: 2, 3, 4, and 5. The value of N in the training data set corresponding to Nₒ has four options of 2869966, 3021293, 11486438, and 25508046. However, it is too difficult for the computational resources of this embodiment to analyze such a large number of ECG signals. Therefore, the upper limit of the number of spliced ECG signals in each category is set to 1,500,000 in the training data set. In the test data set, for each original ECG signal, 20 spliced ECG signals are randomly selected. The prediction summary of the 20 spliced ECG signals is then used as the final prediction of the original ECG signal.

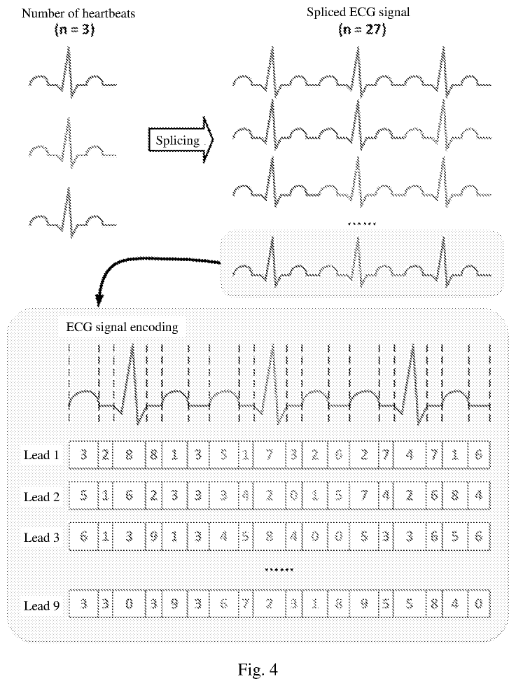

Based on the cluster codes of the segments, the spliced ECG signals can be encoded as respective vectors. Each heartbeat has 6 segments on each lead. In an unsupervised clustering step, each segment is assigned a cluster code. A heartbeat signal can be encoded as a vector with a size of 54 (=6 segments×9 leads). Therefore, the vector size of the spliced ECG signal can be 54×Nₒ. In FIG. 4, the splicing length of Nₒ=3 is taken as an example to demonstrate the process of splicing and encoding 3 original heartbeats. For these 3 original heartbeats, the splicing length of 3 can generate 3³=27 new heartbeat sequences, where each heartbeat has 6 segments with 6 cluster codes. The length of the vector for each new series of heartbeat is N0*6=18.

Each ECG contains 9 leads. The ECG containing 3 heartbeats is finally encoded into 162 (=6 segments×9 leads×3 heartbeats) features.

### 3. Bayesian Classification Model

Based on the encoded features, the spliced ECG signals are classified using Classified Naive Bayes (CNB). Suppose xᵢ (i=1, 2, . . . , n) is the features of ECG, where n is the number of features, and y is the category. According to the conditional probability, the following formula can be obtained:

$$P ( {x}_{1} , {x}_{2} , … , {x}_{n} ) P ( y \vert {x}_{1} , {x}_{2} , … , {x}_{n} ) = P ( y ) p ( {x}_{1} , {x}_{2} , … , {x}_{n} \vert y ) \qquad ( 7 )$$

The above formula can be further deduced as:

$$P ( y \vert {x}_{1} , {x}_{2} , … , {x}_{n} ) = \frac{P ( y ) P ( {x}_{1} , {x}_{2} , … , {x}_{n} \vert y )}{P ( {x}_{1} , {x}_{2} , … , {x}_{n} )} \qquad ( 8 )$$

Based on the Naive Bayesian assumption of feature independence, the following formula can be obtained:

$$p ( {x}_{i} \vert y , {x}_{1} , … , {x}_{i - 1} , {x}_{i + 1} , … , {x}_{i} ) = p ( {x}_{i} \vert y ) \qquad ( 9 )$$

Formula 8 can be further deduced as:

$$P ( y \vert {x}_{1} , {x}_{2} , … , {x}_{n} ) = \frac{P ( y ) {\prod }_{i = 1}^{n} P ( {x}_{i} \vert y )}{P ( {x}_{1} , {x}_{2} , … , {x}_{n} )} \qquad ( 10 )$$

Because P(x₁, x₂, . . . , xₙ) in formula 10 are all constants, the classification rules can be further expressed as follows:

$$\hat{y} = arg max ( P ( y ) {\prod }_{i = 1}^{n} P ( {x}_{i} \vert y ) ) \qquad ( 11 )$$

where ŷ is the category label of the ECG signal, and P(xᵢ|y) can be calculated according to the following formula:

$$P ( {x}_{i} = a \vert y = c ) = \frac{{N}_{i a c}}{{N}_{c}} \qquad ( 12 )$$

where Niac represents the number of spliced ECG signals with the i^{th} feature as category c, Nc represents the number of spliced ECC signals in the category c. The predicted label of the original ECG signal is the average predicted label of the spliced ECG signal.

### 4. Control Model:

In this embodiment, the mode of feature engineering+classifier is used to construct the control model.

Firstly, 114 features are extracted from the ECG signal, where 10 features are defined on 9 separate lead signals (such as the mean value of the P wave peak), such that 90 (10×9) scalar features are obtained. The remaining 24 features are related to the recording time (such as the position in the time dimension) when the segmental key point(s) occurs.

After 114 features are determined, 3 classic classifiers, K nearest neighbor (KNN), random forest (RF) and extreme gradient boosting (XGBoost) are utilized. The 3 classifiers used the respective default configurations thereof.

In addition, two deep learning-based models are also used as control models. The first one is 1D CNN, which achieves state-of-the-art performance in multi-classification of arrhythmia subtypes. The second one is LSTM, which has been proposed for ECG signal classification.

### 5. Quantification and Statistical Analysis

In this embodiment, recall (sensitivity) is used to measure the diagnostic performance of the model for a certain cardiac arrhythmia. In addition, the overall diagnostic performance of the model is measured by macro-recall. Macro-recall herein refers to the recall average across all individual categories. In macro-recall, each class has the same weight when calculating the average recall, instead of setting weights according to the sample size of each class. Thus, macro-recall is fair at the class level and more sensitive to the performance of minority classes. Recalls with 95% confidence intervals are calculated using a nonparametric bootstrap method with 1000 iterations. The linear correlation between recall and sample size or category similarity across 18 categories is measured by correlation coefficient (CC). The CC values can be interpreted as shown below: very weak (0.00-0.19), weak (0.20-0.39), medium (0.40-0.59), strong (0.60-0.79), and very strong (0.80-1.00).

Therefore, arrhythmias can be further classified into strong arrhythmias and weak arrhythmias in computer-aided diagnosis due to differences in sample size and inter-class similarity. Strong arrhythmias crowd out weak arrhythmias, so patients with weak arrhythmias may be underdiagnosed. For this reason, inspired by the diagnostic thinking of cardiologists, the present invention proposes a method for diagnosing arrhythmia by means of combining electrocardiogram segment clustering with the Bayesian theory. The GDPH ECG arrhythmia data set is used to validate the method of the present invention. Through hyperparameter optimization, the best configuration of the method of the present invention in heartbeat splicing and segment clustering is determined.

Compared with other methods, the method of the present invention has comparable performance on strong arrhythmias yet better performance on weak arrhythmias. In addition, with an increase of crowding behavior of strong arrhythmia, the method of the present invention can still accurately diagnose weak arrhythmia.

In recent years, deep learning has performed prominently in ECG interpretation. Its hierarchical structure allows access to higher-level features; in addition, its powerful feature extraction capability help accommodate complex mappings. However, based on the definition of arrhythmia strength, it is difficult for deep learning models to fairly diagnose weak arrhythmias during training.

In this study, possible pathways to protect weak arrhythmias from being crowded out by strong arrhythmias have been explored. The morphological characteristics of ECG signals are key information for cardiologists to diagnose arrhythmias. Inspired by this, the present invention uses segmented clustering to distinguish ECG signals with different morphological features. The arrangement of ECG signals at the beat level can effectively enrich the sample size of weak arrhythmias. On the other hand, splicing multiple heartbeats can increase the dimensionality of the ECG signal compared to a single heartbeat, which may increase the distance of different arrhythmias in the representation space. The method of the present invention can reduce the issue to a certain extent that weak arrhythmias are crowded out by the strong arrhythmias.

An interpretable ECG computer-aided interpretation system will be more trusted by cardiologists and therefore easier to use. In the method of the present invention, features are encoded by segment cluster numbers which have a clear practical meaning. Diagnostic decisions are made based on conditional probabilities of arrhythmia sub-segments. This is a simplified mathematical description of the cardiologist's prior diagnostic experience. Therefore, some morphological features of arrhythmias may be implicit in segmented clusters with higher conditional probabilities. Some of the morphological features found may match current arrhythmia diagnostic criteria. There are also findings that may not match current diagnostic criteria. However, these mismatched findings may hide new diagnostic markers for arrhythmias. The joint conditional probability of multiple fragments may be a viable approach to discover new diagnostic markers.

In summary, compared with the existing technologies, the present invention provides a whole set of systems, platforms and storage media including an electrocardiogram processing method and an intelligent diagnosis algorithm for arrhythmia. The method includes: 1. an algorithm for electrocardiogram interference elimination based on the signal filtering technology; 2. an algorithm for P-QRS-T feature point location and segment segmentation based on extreme cycle iteration; 3. an algorithm for segment control point selection and segment unsupervised classification; 4. an algorithm for cardiac cycle splicing and encoding based on the cutting-splicing data augmentation. The present invention enables the enhancement of raw ECG transformations into modeled codes. The arrhythmia intelligent diagnosis algorithm of the present invention can model the human learning and cognition ability through a Bayesian-based dynamic programming algorithm. It can diagnose 17 types of arrhythmias more accurately than previous technologies. The model also has the ability of single-sample or small-sample learning, and can identify and judge rare or unseen arrhythmias. In addition, it can be generalized in a human-like manner. Finally, the method of the present invention can also be seamlessly connected with multiple systems in the hospital to form a system, platform and storage medium for an electrocardiogram processing method and an intelligent arrhythmia diagnosis algorithm, which can be applied to clinical electrocardiogram diagnosis practice.

In some alternative embodiments, the functions/operations shown in the block diagrams may not be implemented in the specific order shown in the operational diagrams. For example, depending on the specific function/operation involved, two blocks shown in succession may, in fact, be executed substantially concurrently or the blocks may sometimes be executed in a reverse order. Moreover, the embodiments presented and described in the flowcharts of the present invention are provided by way of example in order to provide a more comprehensive understanding of the technology. The disclosed methods are not limited to the operations and logical flow presented herein. Alternative embodiments can be expected that the order of various operations can be altered and that certain sub-operations described as part of larger operations can be performed independently.

Furthermore, the present invention has been described in the context of functional modules. However, it should be understood that, unless otherwise stated to the contrary, one or more of the described functions and/or features may be integrated into a single physical device and/or software module, or one or more functions and/or features may be implemented in separate physical devices or software modules. It is also to be understood that a detailed discussion of the actual implementation of each module is not necessary to understand the present invention. Rather, the actual implementation of the various functional blocks in the devices disclosed herein will be within the ordinary skill of a person skilled in the art, given consideration of the attributes, functions and inter-relationships of the various functional blocks. Accordingly, a person skilled in the art can implement the present invention set forth in the claims using ordinary technologies without undue experimentation. It is also to be understood that the particular concepts disclosed herein are illustrative only and are not intended to limit the scope of the present invention. The scope of the present invention is to be determined by the full scope of the appended claims and their equivalents.

If the functions described above are realized in the form of software function units and sold or used as independent products, they can be stored in a computer-readable storage medium. Based on this understanding, the essence of the technical solutions of the present invention or the parts thereof that contribute to the existing technologies or the parts of the technical solutions can be embodied in the form of a software product. The computer software product can be stored in a storage medium, and may include a number of instructions to enable a computer device (which may be a personal computer, a server, a network device, etc.) to execute all or part of the steps of the methods described in various embodiments of the present invention. The aforementioned storage medium includes: an USB flash disk, a portable hard disk, a read-only memory (ROM), a random access memory (RAM), a magnetic disk, an optical disk, or other types of media that can store program code.

The logics and/or steps described in the flowcharts or otherwise described herein, for example, sequenced listings of executable instructions for implementing logical functions, can be embodied in any computer-readable medium, for use with an instruction execution system, apparatus, or device (such as a computer-based system, a system including a processor, or other systems that can fetch instructions from an instruction execution system, apparatus, or device and execute instructions), or in conjunction with such an instruction execution system, apparatus or device. For the purposes of this specification, a “computer-readable medium” may be any device that can contain, store, communicate, propagate or transmit a program for use in or in conjunction with an instruction execution system, apparatus or device.

More specific examples (or a non-exhaustive list) of the computer-readable medium include the following: an electrical connection (electronic device) with one or more wires, a laptop computer case (magnetic device), a random access memory (RAM), a read-only memory (ROM), an erasable and programmable read-only memory (EPROM or flash memory), a fiber optic device, and a portable compact disc read-only memory (CDROM). In addition, the computer-readable medium may even be paper or another suitable medium, on which the program can be printed, because the program can be obtained electronically, for example by optical scanning of the paper or suitable medium, followed by editing, interpretation or other suitable processing if necessary, and then stored in a computer memory.

It should be understood that various parts of the present invention can be realized by hardware, software, firmware or their combination. In the embodiments described above, various steps or methods may be implemented by software or firmware stored in memory and executed by a suitable instruction execution system. For example, if implemented by hardware, as in other embodiments, the present invention can be implemented by any one or a combination of the following technologies known in the art: discrete logic circuits having logic gates for implementing logic functions on data signals, ASICs with suitable combinational logic gates, programmable gate arrays (PGA), field programmable gate arrays (FPGA), and the like.

In the description of this specification, descriptions with reference to the terms “one embodiment,” “some embodiments,” “an example,” “specific examples,” or “some examples” mean that specific features, structures, materials or characteristics described in connection with the embodiment(s) or example(s) are included in at least one embodiment or example of the present invention. In this specification, schematic representations of the above terms do not necessarily refer to the same embodiment or example. Furthermore, the specific features, structures, materials or characteristics described herein may be combined in any suitable manner in any one or more embodiments or examples.

While certain embodiments of the present invention have been shown and described, it will be understood by a person of ordinary skill in the art that various changes, modifications, substitutions and modifications can be made to the embodiments without departing from the principles and purposes of the present invention. Hence, the scope of the present invention is defined by the claims and their equivalents.

The above are specific descriptions of certain preferred embodiments of the present invention. However, the present invention is not limited to the embodiment described herein. A person skilled in the art can make various equivalent deformations or replacements without departing from the principles of the present invention. These equivalent modifications or replacements are all within the scope defined by the claims of the present application.

1-10. (canceled)

11. A method for analyzing an electrocardiogram (ECG) to detect arrhythmia using machine learning techniques, the method comprising:
training, by one or more processors, a machine learning model to detect arrhythmia using, for a plurality of ECGs for a plurality of patients, (i) encoded electrogram data from the ECG indicating characteristics of each band of a heartbeat, wherein the electrogram data is encoded by clustering each band into a cluster of a plurality of clusters each assigned a cluster number, (ii) an indication of whether the patient has arrhythmia and, for a subset of the plurality of patients that have arrhythmia, (iii) an indication of a type of arrhythmia of a plurality of types of arrhythmia;
obtaining, by the one or more processors, electrogram data from an ECG for a patient;
encoding, by the one or more processors, the electrogram data from the ECG for the patient by clustering each band of the electrogram data; and
applying, by the one or more processors, the encoded electrogram data from the ECG for the patient to the trained machine learning model to diagnose a particular type of arrhythmia for the patient.

12. The method of claim 11, wherein obtaining the electrogram data from the ECG for the patient includes:
locating, by the one or more processors, feature points in the electrogram data according to P-QRS-T characteristic waves for N heartbeats;
segmenting, by the one or more processors, the feature points into bands for each of the N heartbeats;
clustering, by the one or more processors, each band into the cluster of the plurality of clusters using k-means clustering;
encoding, by the one or more processors, each band according to the cluster number assigned to the cluster for the band; and
splicing, by the one or more processors, encoded data for each band to generate a feature vector for the N heartbeats for the ECG,
wherein applying the electrogram data from the ECG for the patient to the trained machine learning model applying the feature vector for the ECG to the trained machine learning model to diagnose the particular type of arrhythmia for the patient.

13. The method of claim 12, wherein locating the feature points includes:
determining, by the one or more processors, a position of an R peak using an R peak detection algorithm;
using, by the one or more processors, a moving window to iteratively query both sides of the R peak;
determining, by the one or more processors, a minimum value in the moving window positioned to the left of the R peak as a position of a Q peak;
determining, by the one or more processors, a minimum value in the moving window positioned to the right of the R peak as a position of an S peak;
performing, by the one or more processors, time window traversal from a QRS complex according to the Q, R, and S peaks to identify a P wave and a T wave, including:
calculating, by the one or more processors, a maximum distance between a starting point and an ending point of an auxiliary line segment at each point in windows to the left and right of the QRS complex according to a boundary detection algorithm based on a local distance transformation to find the starting point and the ending point of the P wave and T wave;
establishing, by the one or more processors, a first time window before a Q start of the QRS complex, and a second time window after an S end of the QRS complex;
detecting, by the one or more processors, a P peak within the first time window using the R peak detection algorithm; and
detecting, by the one or more processors, a T peak within the second time window using the R peak detection algorithm;
decomposing, by the one or more processors, the electrogram data into the N heartbeats according to the feature points; and
dividing, by the one or more processors, each heartbeat of the N heartbeats into 6 bands according to the feature points, wherein the bands include a P wave, P-Q interval, QRS complex, S-T interval, T wave, and T-P interval.

14. The method of claim 12, further comprising:
separately counting, by the one or more processors, a distribution of lengths for each type of band;
calculating, by the one or more processors, an average length of each type of band;
determining, by the one or more processors, a number of control points and a number of indexes for each type of band according to the average length; and
determining, by the one or more processors, a set of features for each band according to the average length, the number of control points, and the number of indexes for the type of band,
wherein clustering each band includes clustering the band into one of the plurality of clusters by comparing the determined set of features for the band to a set of features for each of the plurality of clusters.

15. The method of claim 12, wherein clustering each band using k-means clustering includes:
configuring, by the one or more processors, a set of clusters for a particular type of the band, wherein the particular type of the band is one of a P wave, a P-Q interval, a QRS complex, an S-T interval, a T wave, or a T-P interval;
assigning, by the one or more processors, a number to each cluster in the set of clusters;
computing, by the one or more processors, distances between the band and cluster centroids for the set of clusters using Euclidean distance;
clustering, by the one or more processors, the band into one of the set of clusters according to the distances; and
assigning, by the one or more processors, a cluster number to the band corresponding to the cluster assigned to the band.

16. The method of claim 12, further comprising:
classifying, by the one or more processors, the feature vector for the ECG using naive Bayes.

17. The method of claim 11, wherein training the machine learning model includes:
extracting, by the one or more processors, ECG data samples for the plurality of patients;
filtering, by the one or more processors, the ECG data samples to obtain a training data set and a testing data set;
performing, by the one or more processors, data distribution and class similarity analysis on the training data set and the testing data set to improve a discrimination degree of the electrogram data in the training data set and the testing data set.

18. The method of claim 11, wherein training the machine learning model using the electrogram data for the plurality of patients includes:
obtaining, by the one or more processors, ECG data samples for the plurality of patients;
for each of the ECG data samples:
filtering, by the one or more processors, an electromyography (EMG) signal from the ECG data sample using a Butterworth low-pass filter;
filtering, by the one or more processors, a power frequency interference signal from the ECG data sample using a 50 Hz finite impulse response notch filter with a Kaiser window function; and
filtering, by the one or more processors, ECG baseline drift from the ECG data sample using an infinite impulse response zero-phase shift digital filter; and
training, by the one or more processors, the machine learning model using electrogram data from the filtered ECG data samples.

19. A computing device for analyzing and identifying electrocardiogram, the computing device comprising:
one or more processors; and
a non-transitory computer-readable medium storing instruction thereon, that when executed by the one or more processors, cause the computing device to:
train a machine learning model to detect arrhythmia using, for a plurality of ECGs for a plurality of patients, (i) encoded electrogram data from the ECG indicating characteristics of each band of a heartbeat, wherein the electrogram data is encoded by clustering each band into a cluster of a plurality of clusters each assigned a cluster number, (ii) an indication of whether the patient has arrhythmia and, for a subset of the plurality of patients that have arrhythmia, (iii) an indication of a type of arrhythmia of a plurality of types of arrhythmia;
obtain electrogram data from an ECG for a patient;
encode the electrogram data from the ECG for the patient by clustering each band of the electrogram data; and
apply the encoded electrogram data from the ECG for the patient to the trained machine learning model to diagnose a particular type of arrhythmia for the patient.

20. The computing device of claim 19, wherein to obtain the electrogram data from the ECG for the patient, the instructions cause the computing device to:
locate feature points in the electrogram data according to P-QRS-T characteristic waves for N heartbeats;
segment the feature points into bands for each of the N heartbeats;
cluster each band into the cluster of the plurality of clusters using k-means clustering;
encode each band according to the cluster number assigned to the cluster for the band; and
splice encoded data for each band to generate a feature vector for the N heartbeats for the ECG,
wherein the feature vector for the ECG is applied to the trained machine learning model to diagnose the particular type of arrhythmia for the patient.

21. The computing device of claim 20, wherein to locate the feature points, the instructions cause the computing device to:
determine a position of an R peak using an R peak detection algorithm;
use a moving window to iteratively query both sides of the R peak;
determine a minimum value in the moving window positioned to the left of the R peak as a position of a Q peak;
determine a minimum value in the moving window positioned to the right of the R peak as a position of an S peak;
perform time window traversal from a QRS complex according to the Q, R, and S peaks to identify a P wave and a T wave, including:
calculating a maximum distance between a starting point and an ending point of an auxiliary line segment at each point in windows on the left and right of the QRS complex according to a boundary detection algorithm based on a local distance transformation to find the starting point and the ending point of the P wave and T wave;
establish a first time window before a Q start of the QRS complex, and a second time window after an S end of the QRS complex;
detect a P peak within the first time window using the R peak detection algorithm; and
detect a T peak within the second time window using the R peak detection algorithm;
decompose the electrogram data into the N heartbeats according to the feature points; and
divide each heartbeat of the N heartbeats into 6 bands according to the feature points, wherein the bands include a P wave, P-Q interval, QRS complex, S-T interval, T wave, and T-P interval.

22. The computing device of claim 20, wherein the instructions further cause the computing device to:
separately count a distribution of lengths for each type of band;
calculate an average length of each type of band;
determine a number of control points and a number of indexes for each type of band according to the average length; and
determine a set of features for each band according to the average length, the number of control points, and the index number for the type of band,
wherein to cluster each band, the instructions cause the computing device to cluster the band into one of the plurality of clusters by comparing the determined set of features for the band to a set of features for each of the plurality of clusters.

23. The computing device of claim 20, wherein to cluster each band using k-means clustering, the instructions cause the computing device to:
configure a set of clusters for a particular type of the band, wherein the particular type of the band is one of a P wave, a P-Q interval, a QRS complex, an S-T interval, a T wave, or a T-P interval;
assign a number to each cluster in the set of clusters;
compute distances between the band and cluster centroids for the set of clusters using Euclidean distance;
cluster the band into one of the set of clusters according to the distances; and
assign a cluster number to the band corresponding to the cluster assigned to the band.

24. The computing device of claim 20, wherein the instructions further cause the computing device to:
classify the feature vector for the ECG using naive Bayes.

25. A non-transitory computer-readable medium storing instructions thereon that, when executed by one or more processors, cause the one or more processors to:
train a machine learning model to detect arrhythmia using, for a plurality of ECGs for a plurality of patients, (i) encoded electrogram data from the ECG indicating characteristics of each band of a heartbeat, wherein the electrogram data is encoded by clustering each band into a cluster of a plurality of clusters each assigned a cluster number, (ii) an indication of whether the patient has arrhythmia and, for a subset of the plurality of patients that have arrhythmia, (iii) an indication of a type of arrhythmia of a plurality of types of arrhythmia;
obtain electrogram data from an ECG for a patient;
encode the electrogram data from the ECG for the patient by clustering each band of the electrogram data; and
apply the encoded electrogram data from the ECG for the patient to the trained machine learning model to diagnose a particular type of arrhythmia for the patient.

26. The computer-readable medium of claim 25, wherein to obtain the electrogram data from the ECG for the patient, the instructions cause the one or more processors to:
locate feature points in the electrogram data according to P-QRS-T characteristic waves for N heartbeats;
segment the feature points into bands for each of the N heartbeats;
cluster each band into the cluster of the plurality of clusters using k-means clustering;
encode each band according to the cluster number assigned to the cluster for the band; and
splice encoded data for each band to generate a feature vector for the N heartbeats for the ECG,
wherein the feature vector for the ECG is applied to the trained machine learning model to diagnose the particular type of arrhythmia for the patient.

27. The computer-readable medium of claim 26, wherein to locate the feature points, the instructions cause the one or more processors to:
determine a position of an R peak using an R peak detection algorithm;
use a moving window to iteratively query both sides of the R peak;
determine a minimum value in the moving window positioned to the left of the R peak as a position of a Q peak;
determine a minimum value in the moving window positioned to the right of the R peak as a position of an S peak;
perform time window traversal from a QRS complex according to the Q, R, and S peaks to identify a P wave and a T wave, including:
calculating a maximum distance between a starting point and an ending point of an auxiliary line segment at each point in windows on the left and right of the QRS complex according to a boundary detection algorithm based on a local distance transformation to find the starting point and the ending point of the P wave and T wave;
establish a first time window before a Q start of the QRS complex, and a second time window after an S end of the QRS complex;
detect a P peak within the first time window using the R peak detection algorithm; and
detect a T peak within the second time window using the R peak detection algorithm;
decompose the electrogram data into the N heartbeats according to the feature points; and
divide each heartbeat of the N heartbeats into 6 bands according to the feature points, wherein the bands include a P wave, P-Q interval, QRS complex, S-T interval, T wave, and T-P interval.

28. The computer-readable medium of claim 26, wherein the instructions further cause the one or more processors to:
separately count a distribution of lengths for each type of band;
calculate an average length of each type of band;
determine a number of control points and a number of indexes for each type of band according to the average length; and
determine a set of features for each band according to the average length, the number of control points, and the index number for the type of band,
wherein to cluster each band, the instructions cause the one or more processors to cluster the band into one of the plurality of clusters by comparing the determined set of features for the band to a set of features for each of the plurality of clusters.

29. The computer-readable medium of claim 26, wherein to cluster each band using k-means clustering, the instructions cause the one or more processors to:
configure a set of clusters for a particular type of the band, wherein the particular type of the band is one of a P wave, a P-Q interval, a QRS complex, an S-T interval, a T wave, or a T-P interval;
assign a number to each cluster in the set of clusters;
compute distances between the band and cluster centroids for the set of clusters using Euclidean distance;
cluster the band into one of the set of clusters according to the distances; and
assign a cluster number to the band corresponding to the cluster assigned to the band.

30. The computer-readable medium of claim 26, wherein the instructions further cause the one or more processors to
classify the feature vector for the ECG using naive Bayes.
